(19) United States
(12) Patent Application Publication
(10) Pub. No.: US 2007/0286839 A1
(43) Pub. Date: Dec. 13, 2007

(54) COMPOSITION COMPRISING JOJOBA CARBOXYLATES IN COMBINATION WITH JOJOBA ALCOHOL AND COSMETIC FORMULATION COMPRISING SAME

(76) Inventors: SORAYA ROHDE, TUCSON, AZ (US); AMBER SCHWARTZ, TUCSON, AZ (US); CAROL GILLEN, MIDDLETOWN, NY (US); ENRIQUE ALABATA, TORRANCE, CA (US)

Correspondence Address: DALE F. REGELMAN, LAW OFFICE OF DALE F. REGELMAN, P.C., 4231 SOUTH FREMONT AVENUE, TUCSON, AZ, 85714, US

(21) Appl. No.: 11/758,620

(22) Filed: Jun. 5, 2007

Related U.S. Application Data
(60) Provisional application No. 60/811,230, filed on Jun. 5, 2006.

(51) Int. Cl. A61K 8/97; A61K 36/48

(52) U.S. Cl. 424/74; 424/757

(57) ABSTRACT

A non-aqueous mixture comprising a plurality of jojoba carboxylates in combination with a plurality of jojoba alcohols formed by saponifying jojoba oil. Furthermore, a variety of personal care formulations comprising a plurality of jojoba carboxylates in combination with a plurality of jojoba alcohols are disclosed.

### CROSS REFERENCE TO RELATED APPLICATIONS

This application claims priority from a U.S. Provisional Application having Ser. No. 60/811,230 filed Jun. 5, 2006.

### FIELD OF THE INVENTION

The invention is directed to a non-aqueous mixture comprising a plurality of jojoba carboxylates in combination with a plurality of jojoba alcohols.

### BACKGROUND OF THE INVENTION

It is known in the art to formulate various cosmetic formulations which enhance skin hydration. Such cosmetic formulations include a number of compounds to promote skin hydration. Such cosmetic formulations further include one or more thickeners to boost the viscosity. Such cosmetic formulations further include one or more bases to lower the pH.

What is needed is a naturally-derived formulation that promotes skin hydration, that also increases the viscosity of a cosmetic formulation when disposed therein, and that further adjusts the pH of a cosmetic formulation when disposed therein.

### SUMMARY OF THE INVENTION

Applicants' formulation comprising a plurality of jojoba carboxylates in combination with a plurality of jojoba alcohols promotes skin hydration, increases the viscosity of a cosmetic formulation when disposed therein, and adjusts the pH of a cosmetic formulation when disposed therein.

The invention comprises a non-aqueous mixture comprising a plurality of jojoba carboxylates in combination with a plurality of jojoba alcohols. The invention further comprises a variety of personal care formulations including at least one ingredient selected from the group consisting of glycerin, citric acid, stearic acid, and jojoba oil, wherein each personal care formulation further comprises an amount of from about 1 weight percent to about 25 weight percent of a mixture comprising a plurality of jojoba carboxylates and a plurality of jojoba alcohols. The personal care formulations are selected from the group consisting of shampoos, shampoo conditioners, hair styling gels, hair conditioners, hair reparatives, hair tonics, hair fixatives, hair mousses, bath and shower gels, liquid soaps, moisturizing sprays, makeup, pressed powder formulations, lip products, bath additives, sanitizing wipes, hand sanitizers, premoistened towelettes, skin lotions and creams, shaving creams, and sunscreens.

### BRIEF DESCRIPTION OF THE DRAWINGS

The invention will be better understood from a reading of the following detailed description taken in conjunction with the drawings in which like reference designators are used to designate like elements, and in which:

FIG. 1 graphically shows the results of a 4-hour hydration study using a plurality of topical compositions comprising Applicants' jojoba carboxylate/jojoba alcohol/glycol formulation;

FIG. 2 graphically shows the results of a 24-hour hydration study using a different topical composition comprising Applicants' jojoba carboxylate/jojoba alcohol/glycol formulation;

FIG. 3 graphically shows the viscosities of aqueous solutions comprising different loadings of Applicants' jojoba carboxylate/jojoba alcohol/glycol formulation;

FIG. 4 graphically shows the pH of a 0.25 weight percent aqueous solution of polyacrylic acid comprising different loadings of Applicants' jojoba carboxylate/jojoba alcohol/glycol formulation; and

FIG. 5 graphically shows the viscosity of a 0.25 weight percent aqueous solution of polyacrylic acid comprising different loadings of Applicants' jojoba carboxylate/jojoba alcohol/glycol formulation.

### DETAILED DESCRIPTION OF THE PREFERRED EMBODIMENTS

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Jojoba oil is obtained from the seed of the shrub Simmondsiachinensis which is native to the Sonoran desert. Jojoba oil is a mixture of naturally-occurring compounds obtained from the jojoba seed, sometimes called the jojoba bean. Jojoba seed contains about 50 weight percent of a yellow oil commonly referred to as jojoba oil. In contrast to other vegetable oils which comprise a mixture of triglycerides, jojoba oil comprises a mixture of long-chain esters.

As a first step in formulating Applicants' personal care formulations, a mixture of jojoba oil-derived esters I are saponified using a base II to form a mixture of jojoba carboxylates III and a mixture of jojoba alcohols IV.

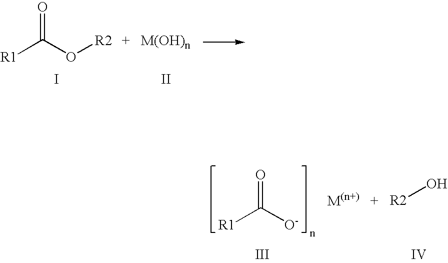

By “jojoba carboxylate,” Applicants means a compound formed by saponifying jojoba oil, wherein that compound comprises a —COO⁻ moiety in combination with a cation. By “jojoba alcohol,” Applicants means a compound formed by saponifying jojoba oil, wherein that compound comprises a hydroxyl moiety.

In certain embodiments, the base II comprises potassium hydroxide, i.e. KOH wherein n equals 1. In other embodiments, base II comprises sodium hydroxide, ammonium hydroxide, magnesium hydroxide, calcium hydroxide, barium hydroxide, zinc hydroxide, and the like.

In certain embodiments, the mixture of esters I comprises naturally-occurring jojoba oil. Jojoba oil includes a variety of ester-group-containing compounds wherein the R1 component comprises a plurality of carbon-containing moieties having, primarily, 17, 18, 20, and 22 carbon atoms, and wherein the R2 component comprises a plurality of carbon-containing moieties having, primarily, 19, 20, 22, and 24 carbon atoms. As those skilled in the art will appreciate, the R1 component of these various jojoba oil ester-group-containing constituents includes at least one carbon-carbon double bond having a cis-configuration. Such a cis-configuration is also known as the Z-configuration. As those skilled in the art will further appreciate, the R2 component of these various jojoba oil ester-group-containing constituents includes at least one carbon-carbon double bond having a cis-configuration. Such a cis-configuration is known as the Z-configuration.

In certain embodiments, mixture of esters I comprises a mixture of “jojoba esters,” wherein those jojoba esters are formed by transesterifying jojoba oil. In certain embodiments, mixture of esters I comprises a mixture of jojoba esters wherein one or both of the double bonds in one or more of the esters have been isomerized to an E, or trans configuration. In certain embodiments, mixture of esters I comprises a plurality of esters wherein one or both of the double bonds have been hydrogenated.

In certain embodiments one or more non-jojoba oil derived hydroxyl group containing compounds are added to the saponification reaction shown above. In certain embodiments, the one or more non-jojoba oil derived hydroxyl group containing compounds comprise an alcohol, such as 1-docosanol sometimes referred to as Behenyl Alcohol and/or propoxylated stearyl alcohol. Table 1 recites a jojoba carboxylate formulation comprising Behenyl Alcohol.

TABLE 1

| COMPONENT | WEIGHT PERCENT |
|---|---|
| JOJOBA ALCOHOLS IV | 20-35 |
| JOJOBA CARBOXYLATES III | 20-35 |
| BEHENYL ALCOHOL | 25-55 |
| RESIDUAL JOJOBA OIL I | <5 |

In certain embodiments, the one or more non-jojoba oil derived hydroxyl group containing compounds comprise a diol, such as propylene glycol, 1,3-butanediol (butylene glycol), and/or 1,4-butanediol. Table 1 recites a jojoba carboxylate formulation comprising butylene glycol. In certain embodiments, the one or more non-jojoba oil derived hydroxyl group containing compounds do not comprise a triol, such as for example and without limitation glycerin.

TABLE 2

| COMPONENT | WEIGHT PERCENT |
|---|---|
| JOJOBA ALCOHOLS IV | 30-45 |
| JOJOBA CARBOXYLATES III | 30-45 |
| BUTYLENE GLYCOL | 5-35 |
| RESIDUAL JOJOBA OIL I | <5 |

Example I is presented to further illustrate to persons skilled in the art how to make and use the invention and to identify certain embodiments thereof. This example is not intended as a limitation, however, upon the scope of the invention.

### EXAMPLE I

Jojoba oil was charged to a clean dry reactor and butylene glycol added with a stoichiometeric amount of potassium hydroxide based upon the ester moieties. This two phase mixture was slowly heated with stirring under nitrogen. After reaching a reaction temperature of between about 35° C. and about 200° C., the reaction mixture was stirred for several hours while that reaction mixture was monitored using Infrared spectroscopy. An ester peak was initially observed, and was seen to disappear. A carboxylate peak appeared and was seen to increase.

After substantially complete conversion of the ester to carboxylate and alcohol the reaction mixture was cooled, and the pH was not adjusted. By “substantially complete conversion,” Applicants mean that less that about 5% of the starting jojoba esters remained. In certain embodiments, the jojobate carboxylate/jojoba alcohol/glycol formulation was bleached with hydrogen peroxide (˜0.1% maximum). An analysis of the jojobate carboxylate/jojoba alcohol/glycol formulation of this Example I showed:
Solids (Potassium Jojobates/Jojoba Alcohols): 80% minimum
Butylene Glycol: 20% maximum
Remaining Ester (by IR): <5%

Applicants' jojobate carboxylate/jojoba alcohol/glycol formulation of Example I was evaluated by MatTek Corporation using an EPIDERM Skin Model In Vitro Toxicity Testing System. More specifically, the jojobate carboxylate/jojoba alcohol/glycol formulation was tested for in vitro dermal irritancy potential using an observed ET-50 test wherein normal, human-derived epidermal keratinocytes were treated with the jojobate carboxylate/jojoba alcohol/glycol formulation of Example I.

The percentage of viable cells was then determined for each test sample using a spectrophotometric analysis at 570 nm. Thereafter, a semi-log plot of the percent viabilities, on the linear Y axis, versus the dosing times, on the log X axis. By interpolation, the time which the percent viability would equal 50%, i.e. the ET-50 time, was determined. A Sodium Lauryl Sulfate control showed an ET-50 time of less than 1 hour. A Triton X control elicited an ET-50 of 3.7 hours. The jojobate carboxylate/jojoba alcohol/glycol formulation of Example I showed an ET-50 time exceeding 24 hours. Applicants' jojobate carboxylate/jojoba alcohol/glycol formulation of Example I is categorized in the non-irritating range.

Applicants' jojobate carboxylate/jojoba alcohol/glycol formulation of Example I was further evaluated using an in vitro EpiOcular irritancy potential using an observed ET-50 test wherein normal, human-derived epidermal keratinocytes which have been cultured to form a stratified, squamous epithelium similar to that found in cornea were treated with the jojobate carboxylate/jojoba alcohol/glycol formulation.

Test cells were treated with Applicants' jojobate carboxylate/jojoba alcohol/glycol formulation for about 5 minutes to about 4 hours. The percentage of viable cells was then determined for each test sample using spectrophotometric analysis at 570 nm. Thereafter, a semi-log plot of the percent viabilities, on the linear Y axis, versus the dosing times, on the log X axis. By interpolation, the time which the percent viability would equal 50%, i.e. the ET-50 time, was determined. Equation 1 can be used to estimate the rabbit Draize eye score:
DRAIZE=−4.74+101.7/(ET-50)^{1/2} (1)

Applicants' jojobate carboxylate/jojoba alcohol/glycol formulation of Example I showed an ET-50 time greater than 256 minutes. That jojobate carboxylate/jojoba alcohol/glycol formulation was given an estimated Draize ocular irritation score of 0.

Applicants have discovered that their jojobate carboxylate/jojoba alcohol/glycol formulation enhances the hydration of human skin. A 4-hour hydration study was performed by Evalulab, 5475 Pare, Suite 206, Mont-Royal, Quebec, Canada. This 4-hour hydration study included a control, a gel base comprising 10 weight percent of the jojobate carboxylate/jojoba alcohol/glycol formulation of Example I, a gel base comprising 15 weight percent of the jojobate carboxylate/jojoba alcohol/glycol formulation of Example I, and a shave cream formulation comprising 10 weight percent of the jojobate carboxylate/jojoba alcohol/glycol formulation of Example I. Table 3 recites the 10 and 15 weight percent jojobate carboxylate/jojoba alcohol/glycol gel formulations. Table 4 recites the shave cream formulation.

TABLE 3

| | 10 WEIGHT PERCENT | 15 WEIGHT PERCENT |
|---|---|---|
| | JOJOBA | JOJOBA |
| COMPONENT | CARBOXYLATE | CARBOXYLATE |
| Water | 88.80 | 83.80 |
| Carbopol | 0.20 | 0.20 |
| Phenoxyethanol | 0.80 | 0.80 |
| Methylparaben | 0.20 | 0.20 |
| Jojoba | 10.00 | 15.00 |
| Carboxylate/Jojoba | | |
| Alcohol/Glycol | | |
| Formulation | | |

TABLE 4

| COMPONENT | WEIGHT PERCENT |
|---|---|
| WATER | 60.8 |
| Butylene Glycol | 1.00 |
| Xanthan Gum | 0.50 |
| Sodium Cocoyl Isethionate | 10.00 |
| Jojoba Carboxylate/Jojoba Alcohol/ | 10.00 |
| Glycol Formulation | |
| Sodium Methyl 2-Sulfoacetate, Disoldium | 8.50 |
| 2-Sulfolaurate, Sodium Lauryl Sulfoacetate | |
| Stearic Acid | 3.00 |
| Cetyl Alcohol | 3.00 |
| Glyceryl Stearate | 2.00 |
| Phenoxyethanol | 0.80 |
| Methylparaben | 0.20 |
| Fragrance | 0.20 |

Skin hydration was determined by measuring the skin capacitance, a parameter related to the electric properties of the corny layer and influenced by the amount of water present. Due to the dipolar nature of water, the higher the hydration level, the higher the conductivity of the skin, resulting in higher measured capacitance.

The study used 12 subjects, each subject having a measured skin hydration level below 30 units. On the day of the study, after 20 minutes of stabilization in a controlled room, the test zones (forearms or lower outer legs) of each subject were examined to ensure a “dry” state skin. After selecting either the forearm or lower outer leg for testing, the selected area was divided into distinct test zones and marked. The test zones were adjacent to each other and measured 9 cm² (3 cm×3 cm) each. The test topical formulations were applied onto the test zones. The test zones were distributed at random per formulation to reduce systematic errors related to anatomical differences between the subjects. The dose applied per test zone was constant, at 2 mg/cm².

Hydration measurements were taken at t=0 (before application), t=30 minutes, t=1 hour, t=2 hours, t=3 hours, and t=4 hours. Hydration was measured using a Corneometer CM 825 sold in commerce by Courage & Khazaka. The Corneometer produces arbitrary units of measurement: <20=very dry, 20-30=dry, 50=normal, etc. Table 5 recites the data collected during this 4-hour hydration study. Each data point comprises an average over the 12 subjects of the study. Control data points were taken from untreated skin areas.

TABLE 5

| | Time | Time | Time | Time | Time | Time |
|---|---|---|---|---|---|---|
| | 0 Hour | 0.5 Hour | 1 Hour | 2 Hour | 3 Hour | 4 Hour |
| Control | 21.25 | 20.75 | 21.42 | 22.17 | 22.08 | 21.83 |
| Shave Cream | 23.08 | 31.50 | 31.83 | 32.50 | 31.42 | 30.92 |
| Base - 10% | 22.58 | 36.08 | 35.67 | 33.50 | 32.92 | 32.50 |
| Jojoba | | | | | | |
| Carboxylate/ | | | | | | |
| Jojoba | | | | | | |
| Alcohol/ | | | | | | |
| Glycol | | | | | | |
| Formulation | | | | | | |
| Base - 15% | 22.25 | 39.67 | 37.00 | 35.08 | 33.25 | 31.83 |
| Jojoba | | | | | | |
| Carboxylate/ | | | | | | |
| Jojoba | | | | | | |
| Alcohol/ | | | | | | |
| Glycol | | | | | | |
| Formulation | | | | | | |

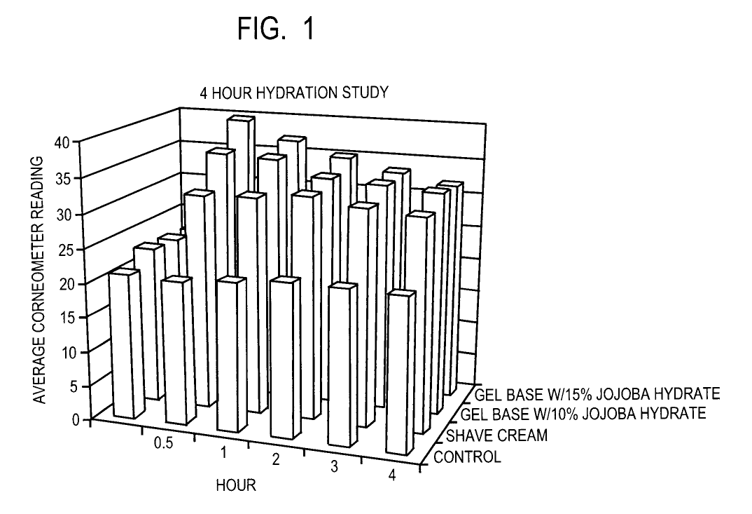

FIG. 1 graphically depicts the data of Table 5. As the data of Table 5 and FIG. 1 show, application of each of the jojoba carboxylate formulations enhanced the measured skin hydration with respect to the control. Moreover, application of the gel base comprising 15 weight percent jojoba carboxylate almost doubled the skin hydration compared to the control at 30 minutes post application.

A 24-hour hydration study was performed by Evalulab, 5475 Pare, Suite 206, Mont-Royal, Quebec, Canada. This 24-hour hydration study included a control, a prior art formulation, and a 23 weight percent in water solution of the Jojoba Carboxylate/Jojoba Alcohol/Glycol formulation of Example I. Table 6 recites the compositions for the compositions examined in the 24-hour study.

TABLE 6

| | | 23 WEIGHT |
|---|---|---|
| | | PERCENT JOJOBA |
| | PRIOR ART | CARBOXYLATE IN |
| COMPONENT | FORMULATION | WATER |
| Water | 98.02 | 76.00 |
| Carbopol | 0.20 | |
| Potassium | 0.78 | |
| Hydroxide | | |
| Phenoxyethanol | 0.80 | 0.80 |
| Methylparaben | 0.20 | 0.20 |
| Jojoba Carboxylate/ | | 23.00 |
| Jojoba Alcohol/ | | |
| Glycol Formulation | | |

The 24 hour hydration study was conducted in accord with the 4 hour study described hereinabove. Hydration measurements were taken at t=0 (before application), t=30 minutes, t=1 hour, t=2 hours, t=3 hours, t=4 hours, and t=24 hours. Hydration was measured using a Corneometer CM 825 sold in commerce by Courage & Khazaka. Table 7 recites the data collected during this first hydration study. Each data point comprises an average over the 12 subjects of the study. Control data points were taken from untreated skin areas.

TABLE 7

| | Time | Time | Time | Time | Time | Time | Time |
|---|---|---|---|---|---|---|---|
| | 0 Hours | 0.5 Hour | 1 Hour | 2 Hours | 3 Hours | 4 Hours | 24 Hours |
| Control | 21.50 | 21.42 | 22.00 | 21.08 | 21.83 | 22.17 | 23.67 |
| Prior art formulation | 22.92 | 32.00 | 33.25 | 34.50 | 35.33 | 35.00 | 33.00 |
| 23% Jojoba | 22.33 | 42.42 | 40.08 | 38.67 | 38.08 | 37.50 | 32.17 |
| Carboxylate/Jojoba | | | | | | | |
| Alcohol/Glycol | | | | | | | |
| Formulation | | | | | | | |

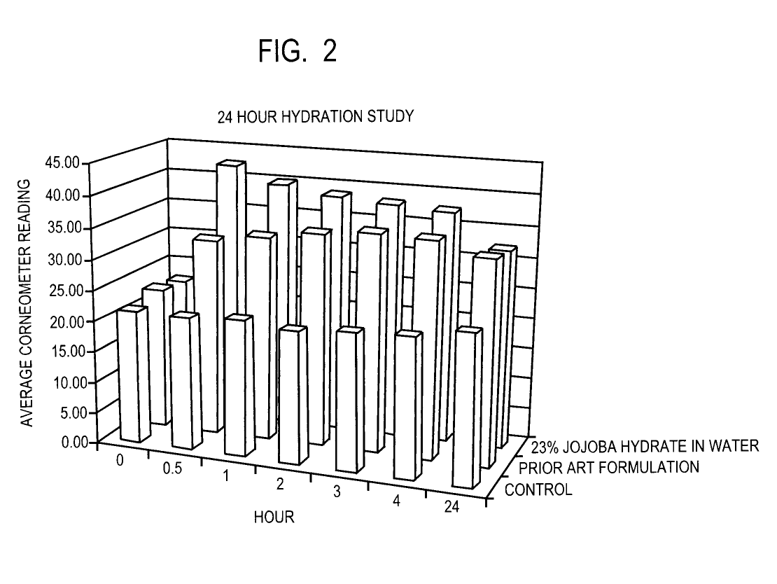

FIG. 2 graphically depicts the data of Table 7. As the data of Table 7 and FIG. 2 show, application of each of the Jojoba Carboxylate/Jojoba Alcohol/Glycol formulation of Example 1 is enhanced the measured skin hydration with respect to the control. Moreover, the enhanced skin hydration was observed for as long as 24 hours after application.

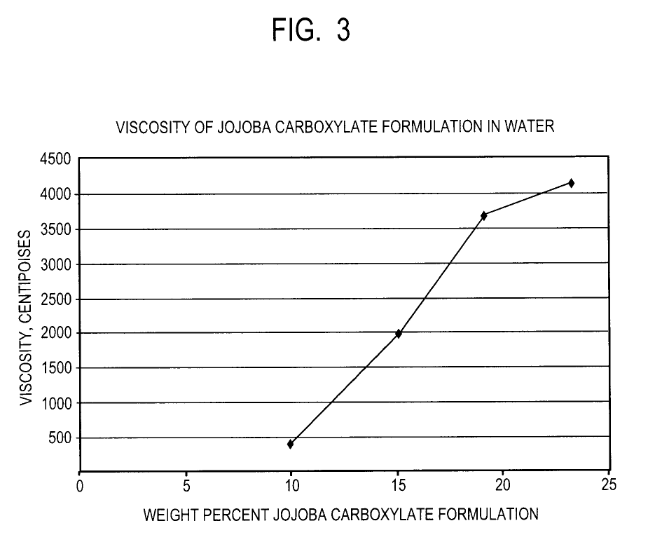

Applicants have found that disposing their Jojoba Carboxylate/Jojoba Alcohol/Glycol formulation of Example 1 in water increases the bulk viscosity of that aqueous mixture. Table 8 recites measured viscosities for various loadings of the Jojoba Carboxylate/Jojoba Alcohol/Glycol formulation of Example 1 in water. FIG. 3 graphically depicts the data of Table 8. The data of Table 8 and FIG. 3 show that the bulk viscosity of an aqueous mixture of Applicants' Jojoba Carboxylate/Jojoba Alcohol formulation increases linearly from a viscosity of 432 cps at a 10 weight percent loading to a viscosity of 4180 cps at a 23 weight percent loading.

TABLE 8

| WEIGHT PERCENT | |
|---|---|
| Jojoba Carboxylate/ | |
| Jojoba Alcohol/ | |
| Glycol Formulation | VISCOSITY, CPS |
| 10 | 432 |
| 15 | 1972 |
| 19 | 3712 |
| 23 | 4180 |

Applicants have further discovered that their Jojoba Carboxylate/Jojoba Alcohol/Glycol formulation of Example 1 can be used to adjust the pH and/or the viscosity of an aqueous solution of polyacrylic acid (“PAA”) V. In certain embodiments, R3 is H and R4 is COOH.

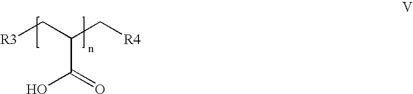

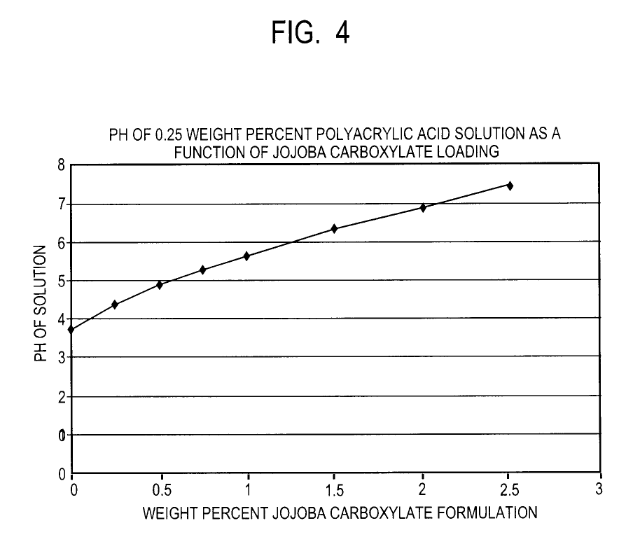

In certain embodiments, PAA V comprises a product sold in commerce by Noveon under the trade name CARBOPOL ULTREZ 10 POLYMER. Table 9 recites measured pH values for a 0.25 weight percent aqueous solution of CARBOPOL ULTREZ 10 POLYMER, wherein that solution further comprises varying amounts of the Jojoba Carboxylate/Jojoba Alcohol/Glycol formulation of Example 1. FIG. 4 graphically depicts the data of Table 9.

TABLE 9

| Weight Percent | |
|---|---|
| Jojoba Carboxylate/ | |
| Jojoba Alcohol/ | |
| Glycol Formulation | pH |
| 0.00 | 3.73 |
| 0.25 | 4.35 |
| 0.50 | 4.87 |
| 0.75 | 5.28 |
| 1.00 | 5.61 |
| 1.50 | 6.31 |
| 2.00 | 6.81 |
| 2.50 | 7.41 |

Referring now to Table 9 and FIG. 4, the pH of a 0.25 weight percent aqueous solution of CARBOPOL ULTREZ 10 POLYMER has a pH of 3.73. The pH of that aqueous solution increases about linearly with the addition of Applicants' Jojoba Carboxylate/Jojoba Alcohol/Glycol formulation of Example 1 to a pH of 7.41 at a loading of 2.5 weight percent of the jojoba carboxylate of Example 1.

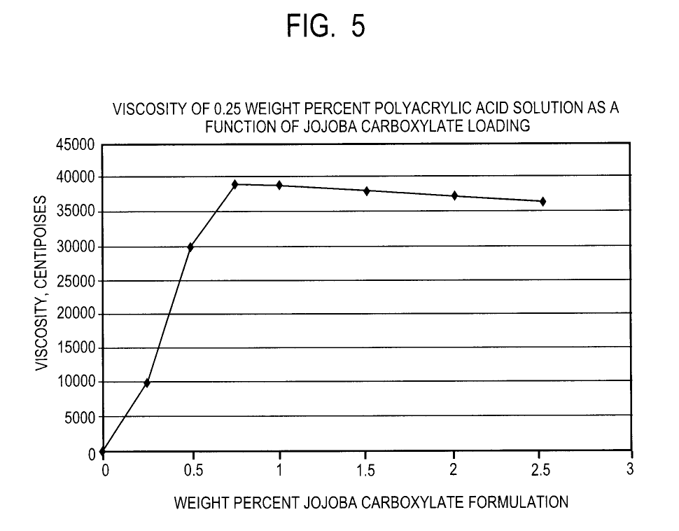

Table 10 recites measured viscosities for a 0.25 weight percent aqueous solution of CARBOPOL ULTREZ 10 POLYMER, wherein that solution further comprises varying amounts of the Jojoba Carboxylate/Jojoba Alcohol/Glycol formulation of Example 1. FIG. 5 graphically depicts the data of Table 10.

TABLE 10

| Weight Percent | |
|---|---|
| Jojoba Carboxylate/ | |
| Jojoba Alcohol/ | |
| Glycol Formulation | Viscosity |
| 0.00 | 1 |
| 0.25 | 9840 |
| 0.50 | 30000 |
| 0.75 | 39000 |
| 1.00 | 39000 |
| 1.50 | 38200 |
| 2.00 | 37500 |
| 2.50 | 36700 |

Referring now to Table 10 and FIG. 5, the viscosity of a 0.25 weight percent aqueous solution of CARBOPOL ULTREZ 10 POLYMER is about 1 centipoises. The viscosity of that aqueous solution increases discontinuously with the addition of Applicants' Jojoba Carboxylate/Jojoba Alcohol/Glycol formulation of Example 1. The viscosity increases linearly to a viscosity of about 39,000 cps for a jojoba carboxylate loading of about 0.75 weight percent. Addition of more jojoba carboxylate formulation to the 0.75 weight percent loading does not result in an increased solution viscosity. Rather, the viscosity plateaus at between about 37,000 cps and about 39,000 cps.

Referring now to Tables 9 and 10 and FIGS. 4 and 5, Applicants have found that their Jojoba Carboxylate/Jojoba Alcohol/Glycol formulation of Example 1 can be used to form an aqueous solution of polyacrylic acid having a viscosity of about 38,000 cps with a pH as low as about 5 or about as high as 7.5.

The invention further comprises a plurality of personal care formulations and/or personal care items (collectively “personal care formulations”), wherein each personal care formulation comprises at least one ingredient selected from the group consisting of glycerin, citric acid, stearic acid, and jojoba oil, wherein each personal care formulation further comprises an amount of from about 1 weight percent to about 25 weight percent of a mixture comprising a plurality of jojoba carboxylates and a plurality of jojoba alcohols. Applicants' personal care formulations are selected from the group consisting of shampoos, shampoo conditioners, hair styling gels, hair conditioners, hair reparatives, hair tonics, hair fixatives, hair mousses, bath and shower gels, liquid soaps, moisturizing sprays, makeup, pressed powder formulations, lip products, bath additives, sanitizing wipes, hand sanitizers, premoistened towelettes, skin lotions and creams, shaving creams, and sunscreens.

The following Examples comprising Applicants' personal care formulations comprising Applicants' Jojoba Carboxylate/Jojoba Alcohol/Glycol Formulation of Example I are presented to further illustrate to persons skilled in the art how to make and use the invention and to identify certain embodiments thereof. These examples are not intended as limitations, however, upon the scope of the invention.

### EXAMPLE II

CONDITIONING SHAMPOO
pH 6.15

| | | |
|---|---|---|
| INCI NAME | TRADE NAME | WT. % |

Phase A

| | | |
|---|---|---|
| Water | Deionized Water | 52.40 |
| Guar Hydroxypropyl-trimonium | JAGUAR C-162¹ | 0.50 |
| Chloride | | |
| Citric Acid | Citric Acid(25% aq. sol) | 1.10 |

Phase B

| | | |
|---|---|---|
| Jojoba Carboxylate/Jojoba Alcohol/ | | 4.00 |
| Glycol Formulation² | | |
| Ammonium Laureth Sulfate | STANDAPOL EA-2³ | 30.00 |

Phase C

| | | |
|---|---|---|
| Cocamidopropyl Hydroxysultaine | CROSULTAINE C-50⁴ | 10.00 |

Phase D

| | | |
|---|---|---|
| Fragrance | GRAPEFRUIT AND | 1.00 |
| | GINGER (5076565)⁵ | |
| Propylparaben/Methylparaben/ | GERMABEN⁶ | 1.00 |
| Diazolidinyl Urea/Propylene Glycol | | |
| | | 100.0 |

Procedure
Add the Phase A water to a suitable vessel. Begin mixing and sprinkle in the Jaguar. Continue mixing and add the Citric Acid. In a separate vessel, combine the STANDAPOL and the JOJOBA HYDRATE BG. Mix and heat to 70 C. Mix until uniform. Add Phase B to Phase A with mixing. Add Phase C to Phases A and B. Continue mixing and cool to 45 C or lower, and add Phase D. Mix until uniform
Suppliers
¹Rhodia
²Desert Whale Jojoba Company
³Care Chemicals
⁴Croda
⁵Aroma Tech
⁶ISP

### EXAMPLE II

HAIR STYLING GEL
pH 9.30

| | | |
|---|---|---|
| INCI NAME | TRADE NAME | WT. % |

Phase A

| | |
|---|---|
| Deionized Water | 20.0 |
| Jojoba Carboxylate/Jojoba Alcohol/Glycol | 25.0 |
| Formulation¹ | |
| Glycerin | 54.0 |

Phase B

| | | |
|---|---|---|
| Phenoxyethanol | Dowanol EPH² | 0.8 |
| Methylparaben | | 0.2 |
| | | 100.0 |

Procedure
Combine Jojoba Carboxylate/Jojoba Alcohol/Glycol Formulation and Glycerin. Heat to 65-75 C with mixing. When batch is uniform and clear, add water and continue mixing. Premix Phase B. Heat to 50 C to dissolve solids. Add to batch and mix until uniform.
Suppliers
¹Desert Whale Jojoba Company
²Dow Chemical

### EXAMPLE IV

MOISTURIZING SHAVE GEL
pH 7.01

| INCI NAME | TRADE NAME | WT. % |
|---|---|---|
| Phase A | | |
| Water | Deionized Water | 60.8 |
| Phase B | | |
| Butylene Glycol | 1,3 Butylene Glycol | 1.0 |
| Xanthan Gum | Keltrol CG¹ | 0.5 |
| Phase C | | |
| Sodium Cocoyl Isethionate | Tauranol I-78²⁰ | 10.0 |
| Jojoba Carboxylate/Jojoba Alcohol/Glycol | | 10.0 |
| Formulation³ | | |
| Sodium Methyl 2-Sulfoacetate (and) | Stepan Mild PCL⁴ | 8.5 |
| Disodium 2-Sulfolaurate (and) Sodium | | |
| Lauryl Sulfoacetate | | |
| Stearic Acid | | 3.0 |
| Cetyl Alcohol | Lipocol C⁵ | 3.0 |
| Glyceryl Stearate SE | Lipo GMS 470⁵ | 2.0 |
| Phase D | | |
| Phenoxyethanol | Dowanol EPH⁶ | 0.8 |
| Methylparaben | | 0.2 |
| Phase E | | |
| Fragrance | Premier S5-29842⁷ | 0.2 |
| | | 100.0 |

Procedure
Heat water to 70-75 C.. Paste Phase B in separate container. Add Phase B to Phase A and mix until uniform and lump-free. At 70-75 C. add Phase C in order. Mix until all solids are dissolved and batch is smooth. Cool to 40 C.. Premix Phase D and warm to 50 C. to dissolve solids. Add to batch and mix. Add Phase E and continue mixing until homogenous.
Suppliers
¹CP Kelco
²Finetex
³Desert Whale Jojoba Company
⁴Stepan
⁵Lipo
⁶Dow Chemicals
⁷Premier

### EXAMPLE V

GEL WITH JOJOBA SPHERES ™
pH 9.77

| INCI NAME | TRADE NAME | WT. % |
|---|---|---|
| Phase A | | |
| Deionized Water | | 74.5 |
| Jojoba Carboxylate/Jojoba Alcohol/ | | 23.0 |
| Glycol Formulation¹ | | |
| Phase B | | |
| Phenoxyethanol | Dowanol EPH² | 0.8 |
| Methylparaben | | 0.2 |
| Phase C | | |
| Hydrogenated Jojoba Oil | LUSH LILAC JOJOBA | 1.5 |
| | SPHERES¹ | |
| | | 100.0 |

Procedure
Heat water to 70-75 C.. Add Jojoba Hydrate and mix until completely dissolved and uniform. In separate container, premix Phase B, warm to 50 C. and mix until all solids are dissolved. Add Phase B to Phase A. Cool batch to below 40 C.. Add Phase C and mix until uniform.
Suppliers
¹Desert Whale Jojoba Company
²Dow Chemical

### EXAMPLE VI

HYDRATING LOTION
A moisturizing lotion comprising Jojoba Carboxylate and Jojoba
Alcohol composition, which provides intense skin hydration due to
a high concentration of jojoba alcohols.

| INCI NAME | TRADE NAME | WT. % |
|---|---|---|
| Phase A | | |
| Distilled Water | | 66.55 |
| Carbomer | Ultrez 21¹ | 0.20 |
| Phase B | | |
| Distilled Water | | 20.00 |
| Tetrasodium EDTA | Versene 100² | 0.05 |
| Jojoba Carboxylate/Jojoba Alcohol/ | | 2.00 |
| Glycol Formulation³ | | |
| Phase C | | |
| Stearic Acid | Emersol 132NF⁴ | 1.00 |
| Glyceryl Monostearate | Stepan GMS Pure⁵ | 2.00 |
| Simmondsia Chinensis (Jojoba) Seed Oil | Jojoba Oil³ | 7.00 |
| Phase D | | |
| Propylene glycol (and) methylparaben | Paragon II⁶ | 1.00 |
| (and) propylparaben (and) DMDM | | |
| hydantion | | |
| Phase E | | |
| Fragrance | Hawaiian Rain⁷ | 0.20 |
| | | 100.0 |

Procedure
1) Sprinkle Ultrez onto water surface and let stand until all particles are wet.
2) Begin mixing until propeller and heat to 70-75 C..
3) Add phase B with propeller.
4) Heat phase C to 80 C. and add to phase AB at 75 C. with propeller mixing.
7) Switch to sweep mixing and add phase D at 45 C..
8) Add phase E at room temperature with sweep mixing.
Suppliers
¹Noveon
²Dow Chemical
³Desert Whale Jojoba Company
⁴Cognis
⁵Stepan
⁶McIntyre
⁷Wellington Fragrance Co.

### EXAMPLE VII

MOISTURIZING CLEANSER
A moisturizing cleanser containing a Jojoba Carboxylate and Jojoba
Alcohol composition which provides intense skin hydration due to a
high concentration of jojoba alcohol.
pH 7.2

| INCI NAME | TRADE NAME | WT. % |
|---|---|---|
| Phase A | | |
| Distilled Water | | 74.30 |
| Acrylates/C10-30 alkyl acrylate | Carbopol Ultrez 21¹ | 0.20 |
| crosspolymer | | |
| Phase B | | |
| Disodium EDTA | Versene 100² | 0.10 |
| Jojoba Carboxylate/Jojoba Alcohol/ | | 3.00 |
| Glycol Formulation³ | | |
| Phase C | | |
| Stearic Acid | Emersol 132NF⁴ | 20.00 |
| Phase D | | |
| Propylene glycol (and) methylparaben | Paragon II⁵ | 1.00 |
| (and) propylparaben (and) DMDM | | |
| hydantion | | |
| Phase E | | |
| Distilled Water | | 1.00 |
| Cyclodextrin (and) Menthol | Lipo CD Menthol⁶ | 0.40 |
| | | 100.0 |

Procedure
1) Sprinkle Ultrez onto water surface and let stand until all particles are wet (no longer white).
2) Begin mixing with propeller and heat to 70-75 C..
3) Add phase B ingredients with propeller mixing.
4) Heat phase C ingredients to 80 C. and add to phase AB at 75 C. with propeller mixing.
5) Switch to sweep at 40 C. and add phase D.
6) Add premixed phase E at room temperature with sweep.
Suppliers
¹Noveon
²Dow Chemical
³Desert Whale Jojoba Company
⁴Cognis
⁵McIntyre
⁶Lipo Chemical

### EXAMPLE VIII

MASSAGE LIQUID
pH 8.0

| INCI NAME | TRADE NAME | WT. % |
|---|---|---|
| Phase A | | |
| Glycerin | | 99.0 |
| Jojoba Carboxylate/Jojoba Alcohol/ | | 1.0 |
| Glycol Formulation¹ | | |
| | | 100.0 |

Procedure
In a separate container, combine Jojoba Carboxylate/Jojoba Alcohol/Glycol Formulation and about 10% Glycerin. Heat to 50-60 C. with mixing. When premix is uniform and clear, add to the remainder of the Glycerin and continue mixing until uniform.
Suppliers
¹Desert Whale Jojoba Company

### EXAMPLE IX

GEL WITH 30% JOJOBA OIL

| INCI NAME | TRADE NAME | WT. % |
|---|---|---|
| Phase A | | |
| Deionized Water | | 52.9 |
| Jojoba Carboxylate/Jojoba Alcohol/Glycol | | 16.1 |
| Formulation¹ | | |
| Phase B | | |
| Simmondsia Chinensis (Jojoba) Seed Oil | JOJOBA OIL¹ | 30.00 |
| Phase C | | |
| Phenoxyethanol | Dowanol EPH² | 0.8 |
| Methylparaben | | 0.2 |
| | | 100.0 |

Procedure
Heat water to 70-75 C.. Add Jojoba Hydrate and mix until completely dissolved and uniform. Slowly add Phase B in small increments with mixing. Continue adding and mixing until batch is uniform. In separate container, premix Phase C, warm to 50 C. and mix until all solids are dissolved. Add Phase C to batch and mix until homogeneous and smooth.
Suppliers
¹Desert Whale Jojoba Company
²Dow Chemical

### EXAMPLE X

GEL WITH 30% APRICOT KERNEL OIL

| INCI NAME | TRADE NAME | WT. % |
|---|---|---|
| Phase A | | |
| Water | Deionized Water | 52.9 |
| Jojoba Carboxylate/Jojoba Alcohol/Glycol | | 16.1 |
| Formulation¹ | | |
| Phase B | | |
| Phenoxyethanol | Dowanol EPH² | 0.8 |
| Methylparaben | | 0.2 |
| Phase C | | |
| Prunus Armeniaca (Apricot) Kernel Oil | APRICOT | 30.0 |
| | KERNEL OIL¹ | |
| | | 100.0 |

Procedure
Combine Phase A and heat to 70-75 C. with mixing. In a separate vessel, combine Phase B, heat to 45-50 C. and mix until all solids are dissolved. Add Phase B to Phase A and mix until uniform. Slowly add Phase C to batch with mixing. Cool to 35 C..
Suppliers
¹Desert Whale Jojoba
²Dow Chemical

### EXAMPLE XI

GEL WITH 30% DIMETHICONE

| INCI NAME | TRADE NAME | WT. % |
|---|---|---|
| Phase A | | |
| Water | Deionized Water | 52.9 |
| Jojoba Carboxylate/Jojoba Alcohol/Glycol | | 16.1 |
| Formulation¹ | | |
| Phase B | | |
| Phenoxyethanol | Dowanol EPH² | 0.8 |
| Methylparaben | | 0.2 |
| Phase C | | |
| Dimethicone | Chemsil DM 100³ | 30.0 |
| | | 100.0 |

Procedure
Combine Phase A and heat to 70-75 C. with mixing. In a separate vessel, combine Phase B, heat to 45-50 C. and mix until all solids are dissolved. Add Phase B to Phase A and mix until uniform. Slowly add Phase C to batch with mixing. Cool to 35 C..
Suppliers
¹Desert Whale Jojoba
²Dow Chemical
³Chemtec

### EXAMPLE XII

GEL WITH 30% CAPRYLIC/CAPRIC TRIGLYCERIDE

| INCI NAME | TRADE NAME | WT. % |
|---|---|---|
| Phase A | | |
| Water | Deionized Water | 52.9 |
| Jojoba Carboxylate/Jojoba Alcohol/Glycol | | 16.1 |
| Formulation¹ | | |
| Phase B | | |
| Phenoxyethanol | Dowanol EPH² | 0.8 |
| Methylparaben | | 0.2 |
| Phase C | | |
| Caprylic/Capric Triglyceride | Lexol GT-865³ | 30.0 |
| | | 100.0 |

Procedure
Combine Phase A and heat to 70-75 C. with mixing. In a separate vessel, combine Phase B, heat to 45-50 C. and mix until all solids are dissolved. Add Phase B to Phase A and mix until uniform. Slowly add Phase C to batch with mixing. Cool to 35 C..
Suppliers
¹Desert Whale Jojoba
²Dow Chemical
³Inolex

While the preferred embodiments of the present invention have been illustrated in detail, it should be apparent that modifications and adaptations to those embodiments may occur to one skilled in the art without departing from the scope of the present invention as set forth in the following claims.

We claim:

1. A non-aqueous mixture, comprising:
a plurality of jojoba carboxylates; and
a plurality of jojoba alcohols.

2. The non-aqueous mixture of claim 1, wherein:
one or more of said plurality of jojoba carboxylates is selected from the group of jojoba carboxylates consisting of 17, 18, 20, and 22 carbon atoms;
one or more of said plurality of jojoba alcohols is selected from the group of jojoba alcohols consisting of 19, 20, 22, and 24 carbon atoms.

3. The non-aqueous mixture of claim 1, further comprising a non-jojoba oil-derived hydroxyl-group containing compound.

4. The non-aqueous mixture of claim 3, wherein said non-jojoba oil-derived hydroxyl-group containing compound is selected from the group consisting of an alcohol and a diol.

5. The non-aqueous mixture of claim 3, wherein said non-jojoba oil-derived hydroxyl-group containing compound comprises butylene glycol.

6. The non-aqueous mixture of claim 5, consisting of:
a plurality of jojoba alcohols at a level of between about 30 weight percent to about 45 weight percent;
a plurality of jojoba carboxylates at a level of between about 30 to about 45 weight percent;
butylene glycol at a level of between about 5 to about 35 weight percent;
jojoba oil at a level of between about 5 to 0 weight percent.

7. The non-aqueous mixture of claim 3, wherein said non-jojoba oil-derived hydroxyl-group containing compound comprises Behenyl Alcohol.

8. The non-aqueous mixture of claim 5, consisting of:
a plurality of jojoba alcohols at a level of between about 20 weight percent to about 35 weight percent;
a plurality of jojoba carboxylates and counterions at a level of between about 20 to about 35 weight percent;
Behenyl Alcohol at a level of between about 25 to about 55 weight percent;
jojoba oil at a level of between about 5 to 0 weight percent.

9. A method to treat dry skin, comprising the steps of:
supplying a first mixture comprising a plurality of jojoba carboxylates in combination with a plurality of jojoba alcohols;
forming a second mixture by disposing said first mixture in water at a level of about 2 weight percent to about 23 weight percent;
topically applying said second mixture to a portion of the skin of a person in need thereof.

10. The method of claim 9, wherein said supplying a first mixture step comprises supplying a first mixture comprising:
one or more jojoba carboxylates selected from the group of jojoba carboxylates consisting of 17, 18, 20, and 22 carbon atoms;
one or more jojoba alcohols selected from the group of jojoba alcohols consisting of 19, 20, 22, and 24 carbon atoms.

11. The method of claim 9, wherein said supplying a first mixture step comprises supplying a first mixture comprising a plurality of jojoba carboxylates, a plurality of jojoba alcohols, and a non-jojoba oil-derived hydroxyl-group containing compound selected from the group consisting of an alcohol and a diol.

12. The method of claim 11, wherein said supplying a first mixture step comprises supplying a first mixture comprising a plurality of jojoba carboxylates, a plurality of jojoba alcohols, and butylene glycol.

13. The method of claim 12, wherein said supplying a first mixture step comprises supplying a first mixture comprising:
a plurality of jojoba alcohols at a level of between about 30 weight percent to about 45 weight percent;
a plurality of jojoba carboxylates and counterions at a level of between about 30 to about 45 weight percent;
butylene glycol at a level of between about 5 to about 35 weight percent;
jojoba oil at a level of between about 5 to 0 weight percent.

14. The method of claim 11, wherein said supplying a first mixture step comprises supplying a first mixture further comprising a plurality of jojoba carboxylates, a plurality of jojoba alcohols, and Behenyl Alcohol.

15. The method of claim 14, wherein said supplying a first mixture step comprises supplying a first mixture comprising:
a plurality of jojoba alcohols at a level of between about 20 weight percent to about 35 weight percent;
a plurality of jojoba carboxylates and counterions at a level of between about 20 to about 35 weight percent;
Behenyl Alcohol at a level of between about 25 to about 55 weight percent;
jojoba oil at a level of between about 5 to 0 weight percent.

16. In a personal care formulation including at least one ingredient selected from the group consisting of glycerin, citric acid, stearic acid, and jojoba oil, the improvement which comprises an amount of from about 1-25% by weight of a mixture comprising a plurality of jojoba carboxylates and a plurality of jojoba alcohols incorporated into the formulation.

17. The personal care formulation of claim 16, wherein:
one or more of said plurality of jojoba carboxylates is selected from the group of carboxylates consisting of 17, 18, 20, and 22 carbon atoms;
one or more of said plurality of jojoba alcohols is selected from the group of alcohols consisting of 19, 20, 22, and 24 carbon atoms.

18. The personal care formulation of claim 17, said mixture further comprising a non-aqueous hydroxyl-group containing compound.

19. The personal care formulation of claim 16, wherein said hydroxyl-group containing compound is selected from the group consisting of an alcohol and a diol.

20. The personal care formulation of claim 16, wherein said personal care formulation is selected from the group consisting of a shampoo, a shampoo conditioner, a hair styling gel, a hair conditioner, a hair reparative, a hair tonic, a hair fixative, a hair mousse, a bath and shower gel, a liquid soap, a moisturizing spray, a makeup, a pressed powder formulation, a lip product, a bath additive, a sanitizing wipe, a hand sanitizer, a premoistened towelette, a skin lotion, a skin cream, a shaving cream, and a sunscreen.
